(19) United States
(12) Patent Application Publication
(10) Pub. No.: US 2015/0272510 A1
(43) Pub. Date: Oct. 1, 2015

(54) SENSOR-ACTIVATED RHYTHM ANALYSIS: A HEURISTIC SYSTEM FOR PREDICTING ARRHYTHMIAS BY TIME-CORRELATED ELECTROCARDIOGRAPHIC AND NON-ELECTROCARDIOGRAPHIC TESTING

(71) Applicant: Sarah Chin, Oakland, CA (US)

(72) Inventor: Sarah Chin, Oakland, CA (US)

(21) Appl. No.: 14/658,101

(22) Filed: Mar. 13, 2015

(51) Int. Cl. A61B 5/00; A61B 5/16; A61B 5/0245; A61B 5/0408; A61B 5/024; A61B 5/11

(52) U.S. Cl. CPC A61B 5/7275; A61B 5/02405; A61B 5/7246; A61B 5/1112; A61B 5/0059; A61B 5/0245; A61B 5/04085; A61B 5/4839; A61B 5/486; A61B 5/165; A61B 5/0006; A61B 5/746; A61B 2562/0219; A61B 2560/0475; A61B 2562/0271; A61B 2562/0204; A61B 2560/0257

(57) ABSTRACT

A method and apparatus for monitoring biosignals (e.g. bioelectrical signals) is disclosed. In one embodiment, a method and apparatus for anticipating a physiological abnormality (e.g. cardiac arrhythmia) is disclosed. In one embodiment, a method and apparatus moves into a high risk mode in anticipation of a physiological abnormality. In one embodiment, non-physiological risk factors may be derived from historic abnormalities (e.g. historic cardiac arrhythmia) of the user, another individual, or a population; and may be correlated with contemporary non-physiological data in order to anticipate a physiological abnormality.

### CLAIM OF PRIORITY

N/A

### FIELD OF TECHNOLOGY

This disclosure relates generally to a technical field of health-related mobile devices that address or monitor biosignals (e.g. bioelectrical signals) and, in one embodiment, to a method and apparatus of anticipating cardiac arrhythmias.

### BACKGROUND

Abnormal heart rhythms (“cardiac arrhythmias”) are common and potentially life-threatening conditions that can affect patients of all demographics. There is a wide range of causes of cardiac arrhythmias, including structural (e.g., abnormal conduction pathways such as Wolff-Parkinson White), toxic (e.g., caffeine and alcohol), metabolic (e.g., high potassium levels), ischemic (e.g., blocked coronary artery), infection, inflammation, and other causes. In addition, various environmental factors may trigger or be associated with arrhythmias, such as diet (e.g., coffee, alcohol, chocolate), tobacco, physical exertion, positional changes, and temperature.

The prevention and treatment of arrhythmias depend on their underlying cause, potential triggers, severity, and symptomotology. Prevention and treatment may include measures to address the underlying cause, as well as specific pharmacologic therapies, transcatheter ablation, transcutaneous cardioversion, and placement of a temporary or permanent cardiac pacemaker and/or defibrillator. Prevention may also entail the avoidance of inciting environmental factors once identified.

While some cardiac arrhythmias may be diagnosed by means of a single electrocardiogram (EKG), detection and characterization of other arrhythmias may be more problematic, requiring real-time electrocardiographic monitoring during an arrhythmia event, which may be transient, short-lived and highly unpredictable. An ambulatory electrocardiographic device, or Holter monitor, may be worn for days at a time in an attempt to record an arrhythmia event, but such a detection window is necessarily finite, and the data recorded by such a device are limited. Therefore, cardiac arrhythmias are often missed by Holter monitors; detecting these rare but serious events requires a great deal of luck. An insertable cardiac monitor may allow for longer-term arrhythmia surveillance, but such a device is invasive, requiring surgical implantation, and the data recorded are limited.

Both the prevention and treatment of arrhythmias are aided by information about the predisposing factors and physical effects surrounding arrhythmias. For example, if certain environmental factors and activities are associated with the onset of arrhythmias, those activities or environmental factors can be eliminated or reduced. If a patient is lightheaded, confused, fatigued, or short-of-breath during an arrhythmia, it is likely that the arrhythmia is hemodynamically significant and demands treatment. Unfortunately, Holter monitoring currently requires a patient to input potentially helpful correlative data, such as location, activity, diet, positional data, and symptoms manually, usually by means of a handwritten patient journal. Such journal entries are inconvenient, inconsistent, incomplete, and unreliable. In addition, patient identity and other data are stored and transmitted separately from the EKG data.

What is needed is a method and mobile apparatus to monitor a user's electrocardiographic patterns, determine correlations between arrhythmias and contemporaneous non-physiological (e.g., environmental, event, condition, circumstance, physical parameter, location, position, orientation, rotation, motion, velocity, acceleration, temperature, barometric pressure, altitude, air quality, luminosity, sound level) and symptomatic data, and assess the ongoing risk of an arrhythmia based on the gathered data. In a broader sense, what is needed is a method and mobile apparatus to monitor a user's biosignal patterns, determine correlations between abnormalities and contemporaneous environmental (e.g. non-physiological) and symptomatic data, and assess the ongoing risk of a physiological disorder based on the gathered data.

### SUMMARY

A method and apparatus of a mobile device is disclosed. In one embodiment an apparatus and method for anticipating and aiding the diagnoses of a cardiac arrhythmia is disclosed. The method of anticipating a cardiac arrhythmia may comprise comparing a non-physiological risk factor 101 with a contemporary non-physiological datum 141 and entering a high risk mode 121 when the non-physiological risk factor 101 is correlated with the contemporary datum 141. As a result, the cardiac arrhythmia may be predicted, prevented, counted, or monitored; or the user may be warned. In one embodiment, the method of anticipating a cardiac arrhythmia may comprise deriving a non-physiological risk factor 101 by correlating an historic abnormality 206 (of the user, another individual, or a group) with an historic non-physiological datum 204 (of the user, another individual, or a group).

Other embodiments will be apparent from the following description and the appended claims.

### BRIEF DESCRIPTION OF THE DRAWINGS

Example embodiments are illustrated by way of example and not limitation in the figures of accompanying drawings, in which like references indicate similar elements and in which:

FIG. 1 is a block diagram illustrating the method of anticipating a physiological disorder (e.g. an arrhythmia) according to one or more embodiments.

FIG. 2 is a block diagram illustrating the method of anticipating a physiological disorder (e.g. an arrhythmia) according to one or more alternative embodiments.

FIG. 3 is a block diagram illustrating the biosignal monitoring apparatus according to one or more embodiments.

Other features of the present embodiments will be apparent from the accompanying Drawings and from the Detailed Description that follows.

### DETAILED DESCRIPTION

In one embodiment an apparatus and method for anticipating and aiding the diagnoses of a cardiac arrhythmia is disclosed. In one embodiment an apparatus and method for anticipating and aiding the diagnosis of a neural disorder is disclosed. In one embodiment an apparatus and method for anticipating and aiding the diagnosis of a neuromuscular disorder is disclosed. In one embodiment an apparatus and method for anticipating and aiding the diagnosis of a gastrointestinal disorder is disclosed. In one embodiment an apparatus and method for anticipating and aiding the diagnosis of a respiratory disorder is disclosed. As used herein a physiological disorder may comprise a pathological or pathophysiological state, condition, or process, or alteration of or deviation from normal physiology (e.g., a neural disorder, a neuromuscular disorder, a respiratory disorder, or a gastrointestinal disorder). In the following description, for the purpose of explanation, numerous details of some embodiments are set forth in order to provide a thorough understanding of the various embodiments.

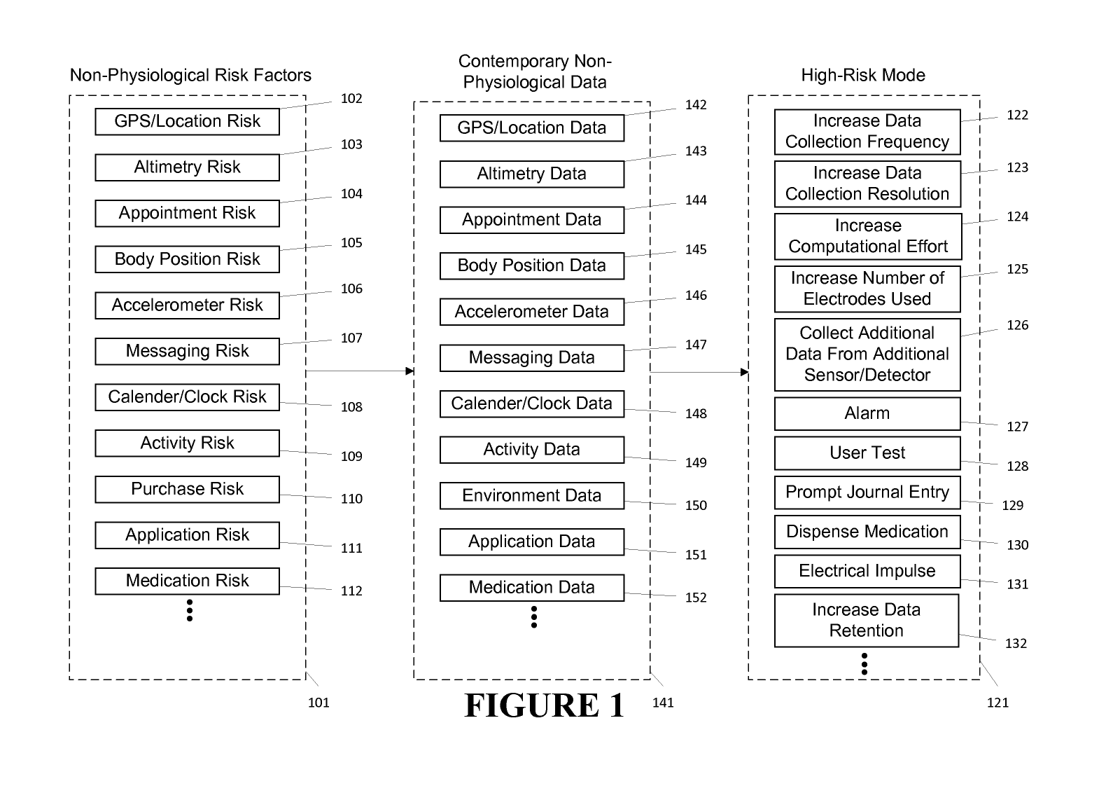

In the embodiment of FIG. 1, the method of anticipating a physiological disorder of a user may comprise comparing with a microcontroller 301 a non-physiological risk factor 101 (of the user, another individual, or a group) with a contemporary non-physiological datum 141 of the user; and entering a high risk mode 121 when the non-physiological risk factor 101 is correlated with the contemporary datum 141. Anticipating a physiological disorder may comprise predicting a physiological disorder such that the physiological disorder may be prevented, counted, or monitored. Anticipating a physiological disorder may comprise predicting a physiological disorder such that the user may be warned, contemporary non-physiological data may be collected, contemporary biosignal data may be collected, other contemporary physiological data may be collected, or a server or other apparatus may be notified. Anticipating a physiological disorder may comprise identifying the physiological disorder as it happens or in advance of the physiological disorder.

A non-physiological risk factor 101 of the user may comprise a risk metric associated with the location or change in location of the user (e.g. GPS location) 102, the altitude or change in altitude of the user (e.g., altimeter, barometer) 103, the nature of the user's activity (e.g. appointment/reminder) 104, the user's body position or change in body position (e.g. gyroscope) 105, the acceleration experienced by the user (e.g. accelerometer) 106, the content of the user's communication (e.g. messaging) 107, the time and/or date (e.g. calendar/clock) 108, the user's activity (e.g., typing, talking) 109, the user's purchases (e.g. caffeine) 110, the user's application history (e.g., browser, calling history, calendar entries, diary entries, activity log, travel log, itinerary, symptom log, food log, address book, media playlist, medication schedule, purchase history) 111, the user's medication history (e.g., medication, OTC meds, alcohol, food) 112, or another risk that may be gathered from the mobile device or deduced from the user's interaction with the mobile device. A non-physiological risk factor 101 of the user may comprise an expectation or probability that a non-physiological factor is correlated with a past or future physiological disorder. A non-physiological risk factor 101 of the user may be derived from an historic physiological disorder (of the user, another individual, or a group); inherent in the device; or entered by a physician, user, or other individual.

A contemporary non-physiological datum 141 of the user may comprise data associated with the location or change in location of the user (e.g. GPS location) 142, the altitude or change in altitude of the user (e.g., altimeter, barometer) 143, the nature of the user's activity (e.g. appointment/reminder) 144, the user's body position or change in body position 145, the acceleration experienced by the user (e.g. accelerometer) 146, the content of the user's communication (e.g. messaging) 147, the time and/or date (e.g. calendar/clock) 148, the user's activity (e.g., typing, talking, purchases) 149, the user's environment (e.g. light/dark) 150, the user's application history (e.g., browser, calling history, calendar entries, diary entries, activity log, travel log, itinerary, symptom log, food log, address book, media playlist, medication schedule, purchase history) 151, the user's medication history (e.g., prescription medication, OTC meds, alcohol, food, drink) 152, or another datum that may be gathered by the mobile device or deduced from the user's interaction with the mobile device.

A high risk mode 121 may comprise one or more modes of the apparatus wherein the apparatus moves from a monitoring mode to a diagnostic mode. A high risk mode 121 may comprise a mode of the apparatus wherein the apparatus moves from a mode of low power consumption to mode of higher power consumption. A high risk mode 121 may comprise a mode of the apparatus wherein data collected by or stored in the apparatus are transmitted to another apparatus or computer. A high risk mode 121 may comprise a mode of the apparatus wherein the apparatus increases data collection frequency 122. A high risk mode 121 may comprise a mode of the apparatus wherein the apparatus increases the data collection resolution 123. A high risk mode may comprise a mode of the apparatus wherein the apparatus increases computational effort 124. A high risk mode may comprise a mode of the apparatus wherein the apparatus increases the number of biosignal electrodes from which data is collected 125. A high risk mode may comprise a mode of the apparatus wherein the apparatus collects additional data from an additional sensor/detector 126. A high risk mode may comprise a mode of the apparatus wherein the apparatus issues an alarm 127, wherein the alarm may comprise an alarm to at least one of the user, another apparatus (e.g. a network computer), and a person (e.g., physician, parent, caretaker, or monitoring service). A high risk mode may comprise a mode of the apparatus wherein the apparatus issues a user test 128, wherein the user test may comprise a test of user consciousness, a test of user mobility, a test of user alertness, a user cognitive test, a test of user health, a test of user status, a test of user position, and a test of user location. A high risk mode may comprise a mode of the apparatus wherein the apparatus prompts the user to make a journal entry 129, wherein the prompt may be at least one of auditory, visual, haptic, vibration, and sensory; and the journal entry may be at least one of auditory, visual, text, motion, touch, and another input to the apparatus. A journal entry may comprise the user's position, activity, location, mental state, health status, or another relevant entry. A journal entry may comprise symptoms (e.g., palpitations, dizziness, syncope, lightheadedness, pain, pallor, flushing, dimming vision, dyspnea, nausea, diaphoresis, anxiety, emotional state, other sensations) recorded by the user or by an observer. A high risk mode may comprise a mode of the apparatus wherein the apparatus dispenses medication or prompts the user to take medication 130. A high risk mode may comprise a mode of the apparatus wherein the apparatus initiates a therapeutic electrical impulse to the user 131. A high risk mode may comprise a mode of the apparatus wherein the apparatus increases the data retention of the apparatus 132. A high risk mode may comprise a mode of the apparatus wherein the apparatus instructs the user to take action (e.g., lie down, locate help, take medication), refrain from action (e.g. stop driving), or control another device (e.g. engage automobile emergency system). Anticipating a physiological disorder may provide the apparatus the benefit of performing in a high risk mode in advance of the physiological disorder.

In one example embodiment, a cardiologist may believe that a user with Wolff-Parkinson-While syndrome tends to enter into a supraventricular tachyarrhythmia when she bends over to tie her shoes. In this example, the user may enter into her smartphone a historic non-physiological risk factor associated with a change in user body attitude from an upright to a bent-over, flexed, semi-prone position, as detectable by sensors of the device (e.g., a multi-axis gyroscope). In this example, if a contemporary non-physiological datum indicates a change in user position from upright to bent-over semi-prone then a high-risk mode may be entered, in which, for example, an audible warning is issued by the device.

In another example embodiment, based on previously determined correlations, a moderate likelihood of entry into atrial fibrillation is predicted, based on location of the user at an ice rink (e.g., GPS sensor, activity log), in the late afternoon, with a current heart rate of 105 bpm, day 8 of predicted menstrual cycle (e.g., personal log), 11 hours since last schedule anti-arrhythmia medication dose (e.g., medication log), ambient temperature 37 F (e.g., thermometer), and upright body position with intermittent 3 g axial load and rotation about z-axis at 120 rpm (e.g., gyroscope and accelerometer).

Cardiac arrhythmias may be detected based on a waveform parameter (e.g., heart rate, P wave length, PR interval, QRS width, QRS axis, RR interval, QT interval, waveform component frequency distribution, or cardiac wave morphology), variability of a waveform parameter, or other derivatives of a waveform parameter. Cardiac arrhythmias may be characterized according to characterization parameters such as time of occurrence, duration, outcome, signal frequency distribution, another waveform parameter, variability of a waveform parameter, or a derivative a waveform parameter. Cardiac arrhythmias may be categorized according to arrhythmia type, such as tachycardia, ventricular tachycardia, supraventricular tachycardia, bradycardia, atrial fibrillation, ventricular fibrillation, conduction delay, aberrant pathway, spatial location of a rhythm variation, direction of propagation of a rhythm variation, or EKG morphology. Cardiac arrhythmias may be reported based on waveform parameters, characterization parameters, arrhythmia type, or non-EKG data of the user (e.g., blood pressure; driving status; location; sleep status). Cardiac arrhythmia reporting may comprise generating a log entry, notification, alert (audible, visible, tactile, and/or palpable), text message, email, phone call, voice message, data transmission, or another communication. The apparatus or method may also report detection of an arrhythmia; determination of a correlation between a detected arrhythmia and other data; an EKG; time and date information; location information; or other data. The apparatus or method may report to the user, a user's relative, a user's physician, an emergency medical response system, a device manufacturer, a monitoring facility, another electronic device, a computer, a network, or other agent or device designated by the user. The apparatus or method may report if a condition is or is not satisfied (e.g. if a user does not respond to a local alert by the device by activating a button or entering a disarm code, then the device may issue an alarm, display emergency medical and contact information on its screen, and/or call 911 and play a pre-recorded message to a 911 operator including the user's identification, location, and condition).

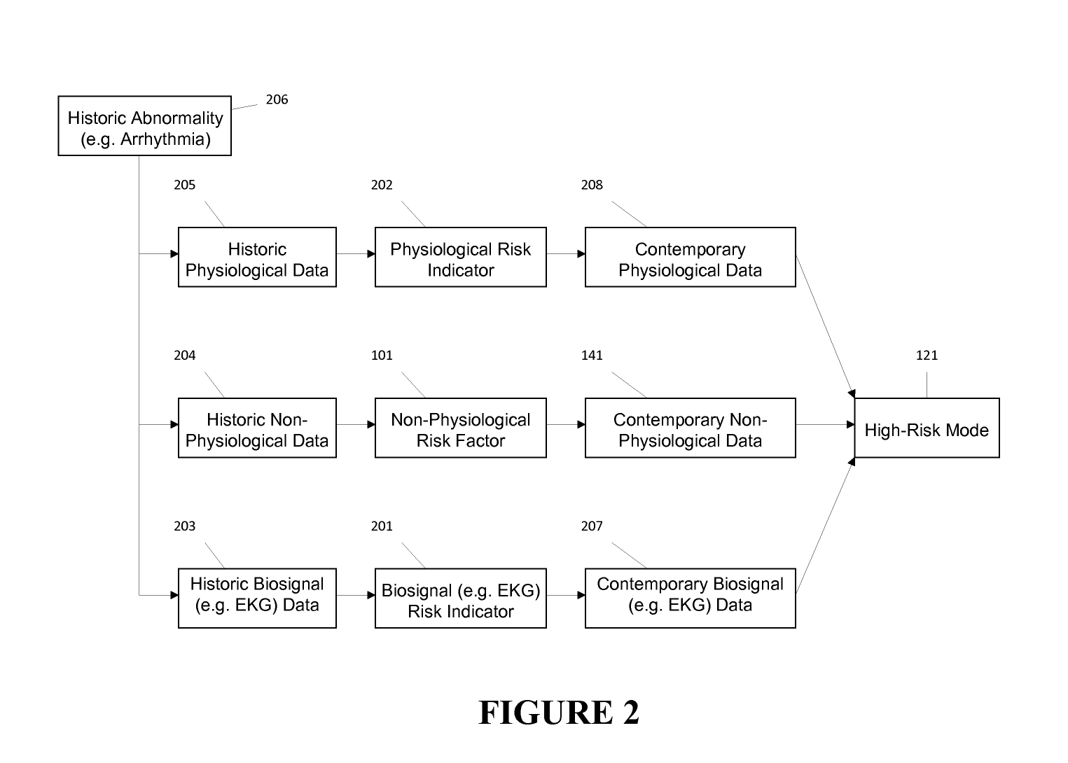

In the embodiment of FIG. 2, the method of anticipating a physiological disorder of a user may comprise deriving with a microcontroller 301 a non-physiological risk factor 101 of the user by correlating an historic abnormality 206 (of the user, another individual, or a group) with an historic non-physiological datum 204, wherein the historic abnormality 206 may be an historic abnormality 206 of the user, another individual, or a group. The method of anticipating a physiological disorder of a user may comprise deriving with a microcontroller 301 a physiological risk indicator 202 of the user by correlating an historic abnormality 206 (of the user, another individual, or a group) with an historic physiological datum 205, wherein the historic abnormality 206 may be an historic abnormality 206 of the user, another individual, or a group. The method of anticipating a physiological disorder of a user may comprise deriving with a microcontroller 301 a biosignal risk indicator 201 of the user by correlating an historic abnormality 206 (of the user, another individual, or a group) with a historic biosignal risk datum 203, wherein the historic abnormality 206 may be an historic abnormality 206 of the user, another individual, or a group.

An historic abnormality 206 may comprise a cardiac arrhythmia, a neural disorder (e.g., a seizure, tremor, motor malfunction, sensory malfunction, psychic malfunction, loss of consciousness, rigidity, stiffness, slowed movement, walking problems, essential tremor, movement malfunction, or depression), a neuromuscular disorder (e.g., muscle weakness, cramping, pain, or contractures), a respiration disorder (e.g., apneic episode, Kussmaul respiration, or Cheyne-Stokes respiration), or a gastrointestinal disorder (e.g. gastrointestinal motility malfunction).

An historic non-physiological datum 204 may comprise a contemporary non-physiological datum 141 that occurred in the past. A historic biosignal datum 203 may comprise a contemporary biosignal datum 207 that was collected in the past. A contemporary biosignal datum 207 may comprise an electrocardiograph (EKG aka ECG) datum, pulse datum, pulse volume datum, respiration datum, pulse oximetry datum, ballistocardiograph datum, another cardiac monitor datum, an electroencephalograph (EEG) datum, an electromyograph (EMG) datum, a nerve conduction datum, or an electrogastroenterograph (EGG or EGEG) datum. A historic physiological datum 205 may comprise a contemporary physiological datum 208 (e.g., respiration rate, pulse rate, body temperature, sweat rate, glucose or insulin level, blood oxygenation level, turgor, electrolyte level, consciousness level, sleep state, skin capacitance, blood medication level, body weight, body mass index, blood pressure, blood test, urine test) that was collected in the past. A non-physiological risk factor 101 may comprise a non-physiological-based (e.g. environmental-based) probability or risk score that indicates a variation in the occurrence risk of a physiological disorder. A physiological risk indicator 202 may comprise a physiological-based probability or risk score that indicates a variation in the occurrence risk of a physiological disorder. A biosignal risk indicator 201 may comprise a biosignal-based probability or risk score that indicates a variation in the occurrence risk of a physiological disorder. A high risk mode 121 may be entered when a contemporary physiological datum is correlated with a physiological risk indicator, when a contemporary non-physiological datum is correlated with a non-physiological risk indicator, or when a contemporary biosignal datum is correlated with a biosignal risk indicator.

In FIG. 2 a contemporary biosignal datum 207 may comprise respiratory patterns monitored by respiratory sensors. Respiration sensors may be worn by or applied to a user (e.g., embedded within a sleeping garment or in a mattress cover) and be designed to detect respiratory activity directly (e.g., by assessing periodic chest or abdominal expansion or by analyzing video data) or indirectly (e.g., by observing variations in transcutaneous pulse oximetry). Specific respiratory patterns may be detected and characterized, such as apneic episodes, Kussmaul respiration (a deep, labored pattern of respiration that may be associated with metabolic acidosis, such as diabetic ketoacidosis), and Cheyne-Stokes respiration (a pattern of waxing and waning deep breaths and regularly recurring apneic periods). Other time-correlated, non-respiratory data (such as user position or orientation, ambient temperature, blood glucose levels, user weight, etc.) collected by, stored in, or accessible to the device may be analyzed in conjunction with the respiratory data and correlations determined. The analyses and correlations may be performed by a microcontroller 301 of the device or by an external computer and/or human expert with access to data. For example, it may be determined that a user's apneic episodes occur most frequently when he is in a supine position and when his BMI (body mass index) exceeds 35.

In FIG. 2 a contemporary biosignal datum 207 or historic biosignal datum 203 may comprise electroenterographic datum (e.g. recordings of electrical signals generated by the neurenteric plexus) or electromyographic data (e.g., activity related to esophageal, gastric, duodenal, enteric, and colonic motility and peristalsis). Such data may be detected, stored, and correlated with other time-aligned data, such as meals (times, volumes, compositions, etc.), medication doses, symptoms, blood chemistry, blood pressure, and other physiologic and environmental data.

In FIG. 2 a contemporary biosignal datum 207 or historic biosignal datum 203 may comprise an electromyographic datum (e.g. recordings of the electrical depolarization of muscle and nerve tissues that may be observed in the setting of nerve compression). In this embodiment carpal tunnel syndrome data may be detected and correlated with other data such as body position, time spent at a computer keyboard, physical activities, diet, symptoms, blood chemistry, and other physiologic and environmental data.

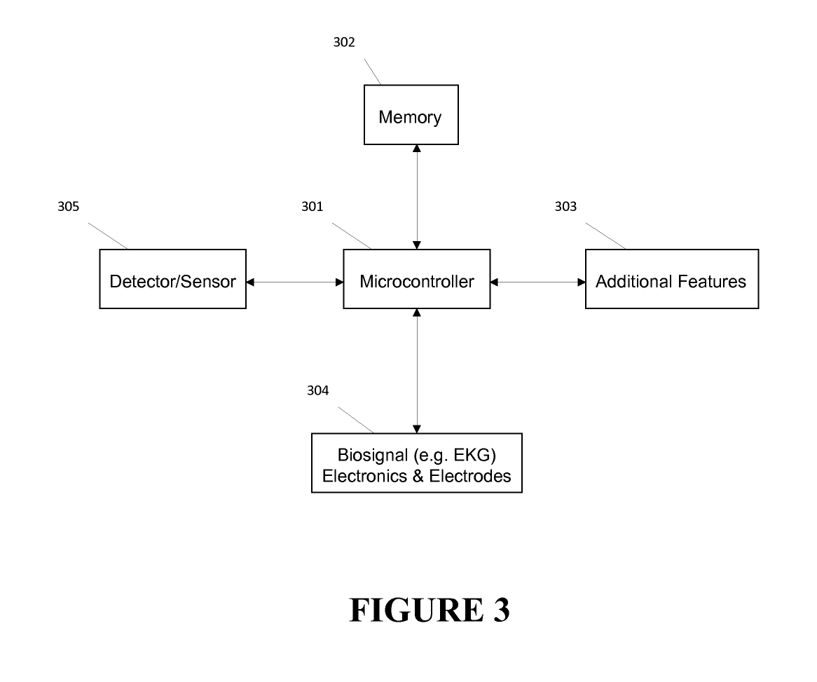

In the embodiment of FIG. 3, the apparatus may comprise a microcontroller 301, a memory 302, biosignal electronics and electrodes 304, one or more detectors/sensors 305, or additional mobile device features 303. Each of these apparatus elements may be coupled, communicatively coupled, or configured to be communicatively coupled to each other element. The microcontroller 301 may comprise a microprocessor, a CPU, a GPU, or another electronic circuit and may be configured to collect and process data of the apparatus. The memory 302 may comprise non-volatile memory, volatile memory, flash, or another electronic storage. The biosignal electronics and electrodes 304 may comprise electrodes for sensing electrical potential (e.g., EKG, EEG, EMG, EGG, EGEG), sensors to measure other physiologic data (e.g., respiration, oxygen saturation, glucose level, electrolyte levels, level of consciousness, sleep state, body temperature, skin capacitance, heart rate, blood pressure, blood medication level, body weight, body mass), amplifiers, filters, converters and another signal processing electronics. The detectors/sensors 305 may comprise an accelerometer, barometric detector, touch sensor, camera, video recorder, light sensor, galvanic sensor, microphone, gyroscopic detector (e.g., single- or multi-axis gyroscope, gyroscopic detector, or gyroscopic sensor), compass, geo-location detector (e.g., local, regional, or global positioning receiver that uses WiFi beacons, a cellular telephone network, or a satellite-based Global Positioning System), microphone, altimeter, barometer, photometer, chronometer, spectrometer, densitometer, chemical sensor, ultrasound emitter or detector, proximity sensor, thermometer, cellular phone network, Wi-Fi transceiver, Bluetooth transceiver, and another electronic sensor. The additional mobile device features 303 may comprise a power supply (e.g. battery), a display screen, a keyboard, a transceiver, a light element, a speaker, a touch sensitive input (e.g. touch screen), a haptic feedback, user interface module, communications module, encryption/decryption module, and another feature of a mobile phone, mobile computer, or mobile network device. The memory 302 may be configured to store a non-physiological risk factor 101, a contemporary non-physiological datum 141, an historic non-physiological datum 204, a physiological risk indicator 202, a contemporary physiological datum 208, an historic physiological datum 205, a biosignal risk indicator 201, a contemporary biosignal datum 207, or an historic biosignal datum 203. The microcontroller may be configured to compare a non-physiological risk factor 101, physiological risk indicator 202, or biosignal risk indicator 201 with a contemporary non-physiological datum 141, contemporary physiological datum 208, or contemporary biosignal datum 207 and enter a high risk mode 121 when a correlation is determined. The microcontroller may be further configured to correlate an historic abnormality 206 with an historic non-physiological datum 204, historic physiological datum 205, or historic biosignal datum 203 and derive or modify (e.g. refine or learn) a non-physiological risk factor 101, physiological risk indicator 202, or biosignal risk indicator 201. The microcontroller may be further configured to encode an historic non-physiological datum 204, an historic physiological datum 205, an historic biosignal datum 203, a contemporary non-physiological datum 141, a contemporary physiological datum 208, or a contemporary biosignal datum 207 with a unique identifier of the user or a cryptographic key. The microcontroller may be further configured to store metadata (e.g., patient identity, patient date of birth, time, location, data source) with the collected data.

A detector/sensor 305 or lead may be positioned percutaneously via a large-bore needle, catheter, sheath, trocar, or cannula wherein a subcutaneous pocket or submuscular cavity may be developed by means of a percutaneously introduced balloon. Such a percutaneous sensor/detector 305 or lead could be positioned in a doctor's office rather than an operating room. A sensor or lead may be coated with a chemical substance (e.g. poly-lactic acid) with the intention of stimulating fibrogenic and other biological tissue ingrowth onto or into the sensor device in order to spatially stabilize the sensor and reduce motion artifact and other noise. A lead may be introduced intra-dermally or sub-dermally as an electro-conductive “tattoo” or thread. A lead may be applied topically as an electro-conductive ink. A sensor or lead may be subcutaneous deployed as a two- or three-dimensional array, for example, to allow collection of spatial and directional data. A noninvasive detector/sensor 305 may comprise a body piercing, tattoo, necklace, eyeglasses, earring, earphone, bracelet, fitness band, anklet, watch, cap, strap, clothing, fabric, mesh, net, garment, applied leads, another wearable item, another item that may be attached to a body, or an item carried by a user.

A biosignal sensor (e.g. EKG) may be separate from, associated with, communicatively coupled with, or housed within a mobile electronic device (e.g., computer, phone, watch, eyewear, network, entertainment, media player, storage, communication, sensor, monitor, camera, or other device). Communicative coupling may be wired or wireless. Biosensor data may be stored in time domain, frequency domain, compressed, encrypted, or another derived form.

In order to mitigate against background noise, one or more additional EKG electrodes may be positioned in a location unlikely to detect significant EKG activity but subject to a background noise signal (e.g., over the right lower back); in this way, background noise may be subtracted or cancelled, increasing overall signal-to-noise ratio. Filtering algorithms may be employed to decrease background noise. Effective signal-to-noise, or statistical power, may also be enhanced by increasing the number of sampled regular or abnormal QRS complexes.

Metadata may be stored and transmitted together with historic physiological data 205, contemporary physiological data 208, historic biosignal data 203, and contemporary biosignal data 207. Historic physiological data 205, contemporary physiological data 208, historic biosignal data 203, and contemporary biosignal data 207 may each be encrypted, either together with or separately from metadata. Historic physiological data 205, contemporary physiological data 208, historic biosignal data 203, and contemporary biosignal data 207 may each be digitally signed, either together with or separately from metadata.

The invention claimed is:

1. A mobile apparatus for anticipating a cardiac arrhythmia of a user, the apparatus comprising:
a microcontroller; and
a memory communicatively coupled to the microcontroller wherein the memory is configured to store a non-physiological risk factor of the user and a contemporary non-physiological datum of the user, wherein
the microcontroller is configured to: compare the risk factor with the contemporary datum, and enter a high risk mode when the risk factor is correlated with the contemporary datum.

2. The apparatus of claim 1 further comprising:
a detector that detects the contemporary datum, wherein the detector is one of:
communicatively coupled to the microcontroller and configured to be communicatively coupled to the microcontroller; wherein
the microcontroller is further configured to collect the contemporary datum from the detector.

3. The apparatus of claim 2 wherein the detector comprises at least one of: an accelerometer, a geo-location receiver, an altimeter, a thermometer, a photometer, a chronometer, a spectrometer, a barometer, a camera, a microphone, and a gyroscopic detector.

4. The apparatus of claim 1 further comprising:
a plurality of electrocardiographic electrodes configured to be applied to the user and configured to be communicatively coupled to the microcontroller, wherein
the microcontroller is further configured to enter the mode when there is a change in an electrocardiograph of the user detected by the microcontroller using a number of the electrodes.

5. The apparatus of claim 4 wherein the mode comprises at least one of: increasing a frequency of data collection of the apparatus, increasing a resolution of data collection of the apparatus, increasing a computational effort of the apparatus, increasing the number of the electrodes used to detect the electrocardiograph, increasing a data retention of the apparatus, dispensing medication to the user, prompting the user to take medication, prompting a user to take action, prompting a user to refrain from action, controlling another device, communicating with another device, transmitting data, and collecting additional data from an additional detector of the apparatus.

6. The apparatus of claim 1 wherein the mode comprises an alarm to at least one of the user, another apparatus, and a person.

7. The apparatus of claim 1 wherein the mode comprises at least one of: a test of user consciousness, a test of user mobility, a test of user alertness, a user cognitive test, a test of user health, a test of user status, a test of user position, and a test of user location.

8. The apparatus of claim 1 wherein the mode comprises prompting the user to journal at least one of their: position, activity, location, mental state, symptom, and health status.

9. The apparatus of claim 1 wherein the microcontroller is further configured to derive the risk factor by correlating an historic arrhythmia with a non-physiological historic datum.

10. The apparatus of claim 1 wherein the microcontroller is further configured to modify the risk factor by correlating an historic arrhythmia with a non-physiological historic datum.

11. A method of anticipating a cardiac arrhythmia of a user the method comprising:
comparing with a microcontroller a non-physiological risk factor of the user with a contemporary non-physiological datum of the user; and
entering a high risk mode when the risk factor is correlated with the contemporary datum.

12. The method of claim 11 further comprising:
collecting the contemporary datum from a detector communicatively coupled with the microcontroller.

13. The method of claim 12 wherein the detector comprises at least one of: an accelerometer, a geo-location receiver, an altimeter, a thermometer, a photometer, a chronometer, a spectrometer, a barometer, a camera, a microphone, and a gyroscopic detector.

14. The method of claim 11 further comprising:
monitoring an electrocardiograph of the user using a number of a plurality of electrocardiographic electrodes;
detecting a change in the electrocardiograph;
entering the high risk mode based on the change in the electrocardiograph.

15. The method of claim 14 wherein the mode comprises at least one of: increasing a frequency of data collection, increasing a resolution of data collection, increasing a computational effort, increasing the number of the electrodes used to detect the electrocardiograph, increasing a data retention, dispensing medication to the user, prompting the user to take medication, prompting a user to take action, prompting a user to refrain from action, controlling another device, communicating with another device, transmitting data, and collecting an additional data from an additional detector.

16. The method of claim 11 wherein the mode comprises an alarm to at least one of the user, another apparatus, and a person.

17. The method of claim 11 wherein the mode comprises at least one of: a test of user consciousness, a test of user mobility, a test of user alertness, a user cognitive test, a test of user health, a test of user status, a test of user position, and a test of user location.

18. The method of claim 11 wherein the mode comprises prompting the user to journal at least one of their: position, activity, location, mental state, symptom, and health status.

19. The method of claim 11 wherein the risk factor is derived by correlating an historic arrhythmia with a non-physiological historic datum.

20. The method of claim 11 wherein the risk factor is modified by correlating an historic arrhythmia with a non-physiological historic datum.
